(19) United States
(12) Patent Application Publication
(10) Pub. No.: US 2007/0149855 A1
(43) Pub. Date: Jun. 28, 2007

(54) ENDOSCOPE

(76) Inventors: Toshiaki NOGUCHI, Tokyo (JP); Sumihiro UCHIMURA, Sagamihara-shi (JP); Tatsuya FURUKAWA, Tokyo (JP); Masahiro KAWAUCHI, Tokyo (JP); Fumiyuki ONODA, Tokyo (JP); Hiroki MORIYAMA, Tokyo (JP); Ryuichi TOYAMA, Tokyo (JP)

Correspondence Address: OSTROLENK FABER GERB & SOFFEN, 1180 AVENUE OF THE AMERICAS, NEW YORK, NY, 100368403, US

(21) Appl. No.: 11/683,182

(22) Filed: Mar. 7, 2007

Related U.S. Application Data
(63) Continuation of application No. PCT/JP05/16233, filed on Sep. 5, 2005.

(30) Foreign Application Priority Data
Sep. 8, 2004 (JP) 2004-261431

(51) Int. Cl. A61B 1/06

(52) U.S. Cl. 600/168

(57) ABSTRACT

An endoscope includes an image capturing unit which is provided with at least a plurality of optical lenses, a zoom lens frame for supporting the optical lenses for zooming out of the plurality of optical lenses, and a first actuator for moving forward and reversely the zoom lens frame in an optical axis direction. The first actuator extends or contracts in accordance with an interruption of power supply.

### CROSS REFERENCE TO RELATED APPLICATION

This application is a continuation application of PCT/JP 2005/016233 filed on Sep. 5, 2005 and claims benefit of Japanese Application No. 2004-261431 filed in Japan on Sep. 8, 2004, the entire contents of which are incorporated herein by this reference.

### BACKGROUND OF THE INVENTION

1. Field of the Invention

The present invention relates to an endoscope which has an image capturing unit provided with a zoom lens.

2. Description of the Related Art

Conventionally, endoscopes which are provided with a zoom lens in an image capturing optical system provided at a tip of an endoscope insertion part have been known. Observers, by moving the zoom lens forward and reversely in an optical axis direction, can obtain an enlarged image or a wide-angle image.

The zoom lens is, generally, held by a zoom lens frame. The zoom lens, in conjunction with the movement of the zoom lens frame in the optical axis direction, moves forward and reversely in the optical axis direction. The zoom lens frame is, when an operation lever, or the like provided in an endoscope operation part is operated, operated forward and reversely by a motor, ultrasonic actuator, a piezo element, or the like provided in the vicinity of the zoom lens frame.

Japanese Unexamined Patent Application Publication No. 2002-122795 proposes a technique that by an operation lever, or the like provided in an endoscope operation part is operated, using a wire connected to the operation lever at one end and connected to a zoom lens frame at the other end, the zoom lens frame is moved forward and reversely in an optical axis direction.

### SUMMARY OF THE INVENTION

An endoscope according to the present invention includes an image capturing unit which is provided with at least a plurality of lenses, a zoom lens frame for supporting the optical lenses for zooming out of the plurality of lenses, and drive means for moving forward and reversely the zoom lens frame in an optical axis direction. The drive means has a first actuator which extends or contracts in accordance with an interruption of power supply.

### BRIEF DESCRIPTION OF THE DRAWINGS

FIG. 1 is a view schematically illustrating a structure of an image capturing unit provided at a tip of an insertion part of an endoscope illustrating a first embodiment of the present invention.

FIG. 2 is a front elevational view of an image capturing unit illustrating a state that a zoom lens frame of FIG. 1 is inserted into a groove of the image capturing unit viewed from the front in an optical axis direction.

FIG. 3 is an enlarged perspective view of a first actuator of FIG. 1.

FIG. 4 is a cross sectional view taken along the line IV-IV of FIG. 3.

FIG. 5 is a partly cross sectional view taken along the line V-V of FIG. 2.

FIG. 6 is a view schematically illustrating a structure of a movement mechanism of a zoom lens frame of an image capturing unit provided at a tip of an insertion part of an endoscope illustrating a second embodiment of the present invention.

FIG. 7 is a perspective view illustrating a conventional forward/reverse drive mechanism of a zoom lens frame.

FIG. 8 is a view schematically illustrating a structure of an image capturing unit provided at a tip of an insertion part of an endoscope illustrating a third embodiment of the present invention.

FIG. 9 is an enlarged front view of a first aperture of FIG. 8.

FIG. 10 is a cross sectional view illustrating a structure of a first actuator of the first aperture of FIG. 8.

FIG. 11A is a view illustrating a state that the first aperture and a second aperture of FIG. 8 contract.

FIG. 11B is a view illustrating a state that the first aperture and the second aperture of FIG. 8 extend.

FIG. 12 is a view illustrating a structure of an end cap attached to an endoscope insertion part tip.

FIG. 13 is a cross sectional view taken along the line XIII-XIII of FIG. 12.

FIG. 14 is a view illustrating a modification that a power supply ring member of FIG. 12 is formed of a plurality of regions.

FIG. 15 is a view illustrating another structure of the end cap attached to the endoscope insertion part tip.

### DESCRIPTION OF THE PREFERRED EMBODIMENTS

Hereinafter, embodiments of the present invention will be described with reference to the drawings.

### First Embodiment

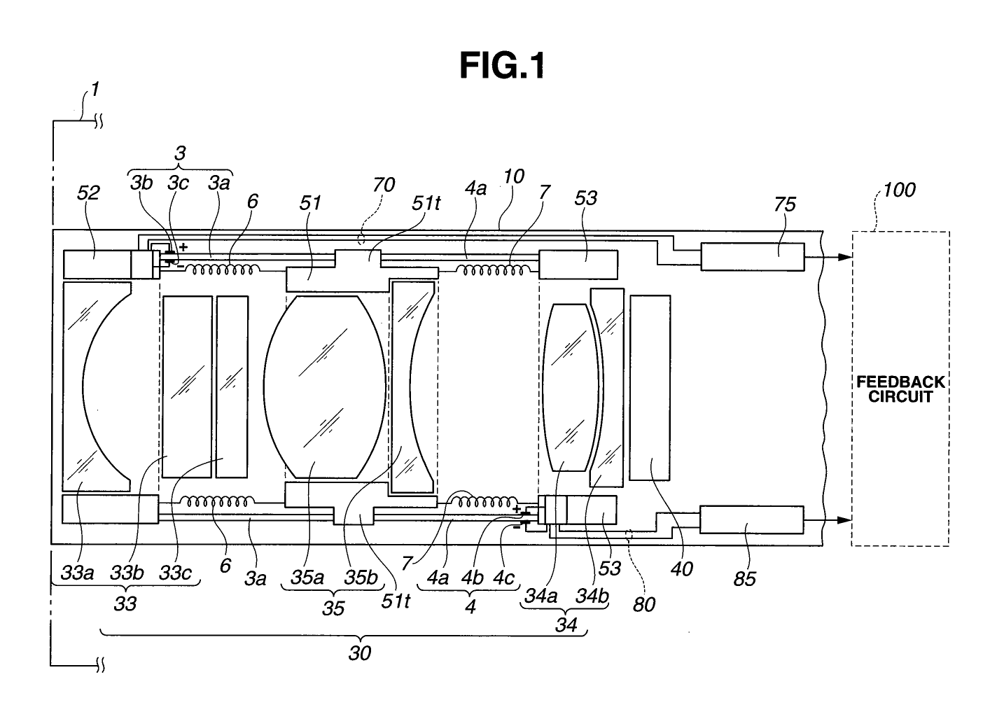

FIG. 1 is a view schematically illustrating a structure of an image capturing unit provided at a tip of an insertion part of an endoscope illustrating the first embodiment of the present invention.

As illustrated in FIG. 1, in an image capturing unit 10 of an endoscope 1, an image capturing optical system 30 which has a plurality of optical lenses is provided. At an optical axis direction base end side (hereinafter, simply referred to as a base end side) of the image capturing optical system 30, an image capturing element 40 such as a CCD on which a subject image received by the image capturing optical system 30 is formed is provided.

The image capturing optical system 30 includes a tip side lens group 33 which is arranged at an optical axis direction tip side (hereinafter, simply referred to as a tip side) of the image capturing unit 10, a base end side lens group 34 which is arranged at the base end side of the image capturing unit 10 and in the vicinity of the tip of the image capturing element 40, and a zoom lens group 35 which is arranged between the tip side lens group 33 and the base end side lens group 34.

The tip side lens group 33 is a fixed lens system which includes, for example, a plurality of optical lenses 33a, 33b, and 33c, and the optical lenses 33a, 33b, and 33c are held by a lens frame (not shown) fixed to the tip side of the image capturing unit 10.

The base end side lens group 34 is a fixed lens system which includes, for example, a plurality of optical lenses 34a and 34b, and the optical lenses 34a and 34b are held by a lens frame (not shown) fixed to the base end side of the image capturing unit 10.

The zoom lens group 35 includes, for example, optical lenses 35a and 35b which are a plurality of zoom lenses, is held by a zoom lens frame 51, and moves forward and reversely between the tip side lens group 33 and the base end side lens group 34 in the optical axis direction.

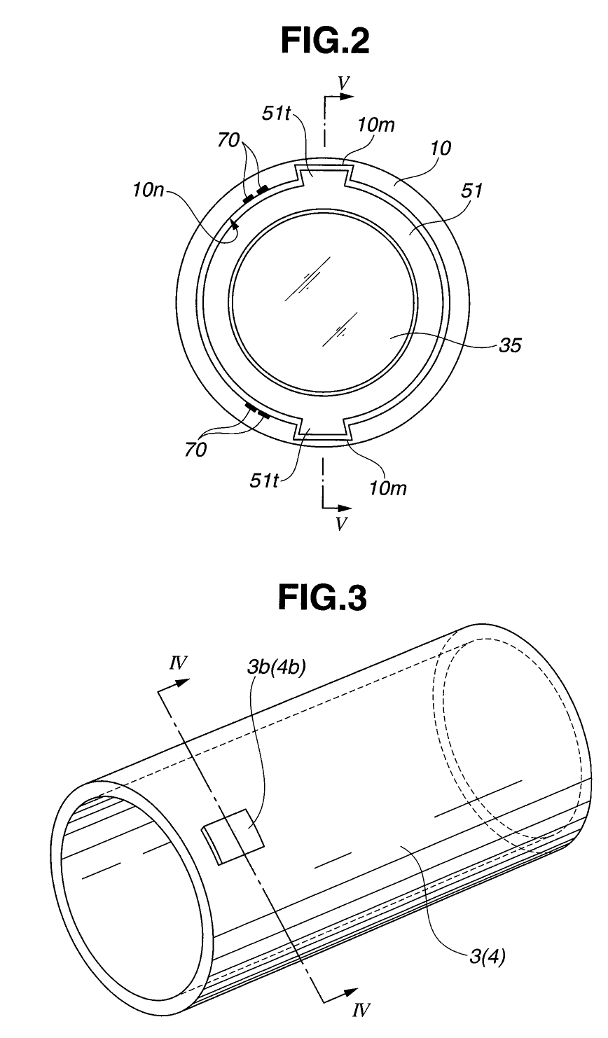

In FIG. 2, a front elevational view of the image capturing unit illustrating a state that the zoom lens frame of FIG. 1 is inserted into a groove of the image capturing unit viewed from the front in the optical axis direction is illustrated.

As illustrated in the drawing, in the zoom lens frame 51, two lens sliding convex parts 5 it which protrude in up and down direction substantially orthogonal in the optical axis direction in the drawing respectively are formed.

Further, on an inner circumference surface 10n of the image capturing unit 10, for example, in up and down direction substantially orthogonal in the optical axis direction in the drawing, two lens sliding grooves 10m into which the two lens sliding convex parts 51t are inserted and slide are formed respectively along the optical axis direction.

Accordingly, the zoom lens frame 51, by the two lens sliding convex parts 51t slide in the lens sliding grooves 10m in the optical axis direction, moves forward and reversely in the optical axis direction being guided by the lens sliding grooves 10m. The guides of the zoom lens frame 51 are not limited to the above, for example, rail can be used.

Returning to FIG. 1, in the vicinity of the outer circumference of the optical lens 33a of the tip side lens group 33, a cylindrical first supporting member 52 is provided. Between the base end surface of the first supporting member 52 and the tip surface of the lens sliding convex part 51t of the zoom lens frame 51, a first actuator 3 which is a drive means which contracts when electric power is supplied is provided.

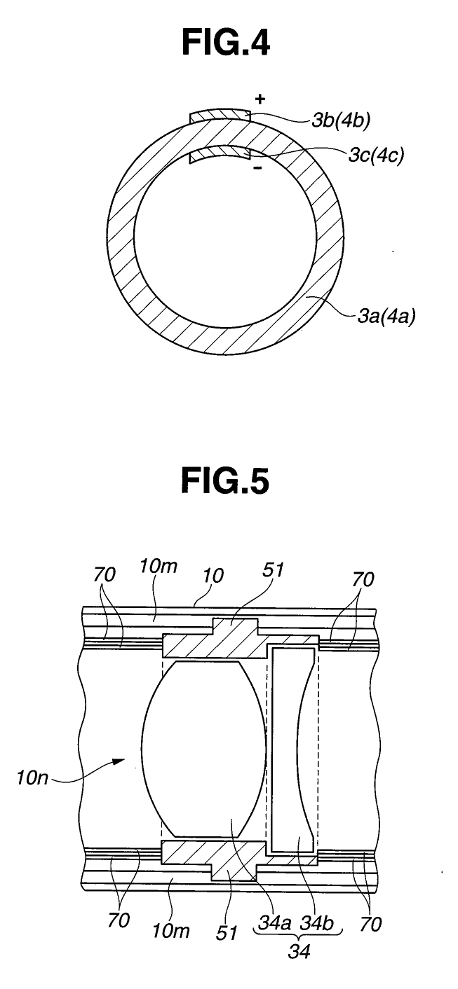

Specifically, in FIG. 3, an enlarged perspective view of the first actuator of FIG. 1, and in FIG. 4, a cross sectional view taken along the line IV-IV of FIG. 3 are illustrated. As illustrated in FIG. 3, the first actuator 3 is formed to have a substantially cylindrical shape.

The first actuator 3 is, by the tip end surface in the optical axis direction is connected to the base end surface of the first supporting member 52, held to be substantially parallel to the first supporting member 52 in the optical axis direction. The base end surfaces of the first actuators 3 in the optical axis direction are connected to the tip surfaces of the two lens sliding convex parts 51t of the zoom lens frames 51.

Further, as illustrated in FIG. 4, the first actuator 3 includes a first polymeric material (hereinafter, referred to as EPAM) 3a formed of a polymeric raw material, for example, so called an ELECTROACTIVIVE POLYMERS AS ARTIFICIAL MUSCLES (EPAM) which extends or contracts in accordance with an interruption of power supply, and a first electrode part which sandwiches at least a part of the first EPAM 3a, and has a plus electrode 3b and a minus electrode 3c which have different polar characters and formed of a polymeric raw material, for example, a conductive rubber.

Accordingly, the first actuator 3 is so called a polymer actuator. The first actuator 3 has a characteristic to extend if the actuator has contracted when the power supply is interrupted. A rate of contraction of the first EPAM 3a and rates of contraction of the plus electrode 3b and the minus electrode 3c are substantially the same.

To the plus electrode 3b and the minus electrode 3c, for example, through a connection wire 70 which is a first signal wire of a lead wire, electric power is supplied from an actuator drive circuit 75 which is a control means provided at the base end side of the image capturing unit 10.

The connection wire 70 is arranged in the vicinity of the inner circumference surface 10n of the image capturing unit 10. One end of the connection wire 70 is electrically connected to the electrodes 3b and 3c, and the other end is connected to the actuator drive circuit 75.

The actuator drive circuit 75, in response to a signal from a feedback circuit 100 which is a control means of the endoscope 1, supplies electric power to the first actuator 3.

The actuator drive circuit 75 can be provided in an operation part (not shown) of the endoscope 1, a video processor, or the like connected to the endoscope 1. Thus, the first EPAM 3a contracts in the optical axis direction and the zoom lens frame 51 to which the base end surface of the first actuator 3 is connected moves forward in the optical axis direction.

The connection wire 70 which transmits the electric power to the plus electrode 3b and the minus electrode 3c from the actuator drive circuit 75 is not necessary to be the lead wire, but the connection wire can be, for example, formed of an electric pattern structure.

Specifically, FIG. 5 is a partly cross sectional view taken along the line V-V of FIG. 2, and as illustrated in the drawing, the connection wire 70 can be formed on the inner circumference surface 10n of the image capturing unit 10 by coating or etching. Thus, the inner structure of the image capturing unit 10 can be simplified.

Returning to FIG. 1, between the base end surface of the first supporting member 52 and the tip surface of the zoom lens frame 51, and in the vicinity of the inner circumference of the first actuator 3, two elastic members 6, for example, coil springs, are provided. The elastic members 6 are not limited to the coil springs, but can be springs or rubber.

The elastic members 6 return the position of the moved zoom lens frame 51 in the optical axis direction to the position before the movement. Specifically, elastic members 6 maintain the position of the zoom lens frame 51 in the optical axis direction of the time the electric power is not supplied to the first actuator 3.

Returning to FIG. 1, in the vicinity of the outer circumference of the base end side lens group 34, a cylindrical second supporting member 53 is provided. Between the tip surface of the second supporting member 53 and the base end surface of the lens sliding convex part 51t of the zoom lens frame 51, a second actuator 4 which is a position detection means which generates electric power by deformation is provided.

Specifically, the second actuator 4 is formed to have, similarly to the first actuator 3, as illustrated in FIG. 3, a substantially cylindrical shape. The second actuator 4 is, by the base end surface in the optical axis direction is connected to the tip surface of the second supporting member 53, held to be substantially parallel to the second supporting member 53 in the optical axis direction. The tip surface of the second actuator 4 in the optical axis direction is connected to the base end surface of the lens sliding convex parts 51t of the zoom lens frame 51.

Further, as illustrated in FIG. 4, the second actuator 4 includes a second EPAM 4a formed of a polymeric raw material, for example, an EPAM which generates by deformation, and a second electrode part which sandwiches at least a part of the second EPAM 4a, and has two electrodes, that is, a plus electrode 4b and a minus electrode 4c which are formed of a polymeric raw material, for example, a conductive rubber, and have different polar characters.

Accordingly, the second actuator 4 is so called a polymer actuator. A rate of deformation of the second EPAM 4a and rates of deformation of the plus electrode 4b and the minus electrode 4c are substantially the same.

To the plus electrode 4b and the minus electrode 4c, one end of a connection wire 80 which is a second signal wire of a lead wire, for example, is electrically connected. The other end of the connection wire 80 is connected to a position detection processing circuit 85 which is a control means provided at the base end side of the image capturing unit 10.

The connection wire 80 is provided in the vicinity of the inner circumference surface 10n of the image capturing unit 10. The position detection processing circuit 85 can be provided in an operation part (not shown) of the endoscope 1, a video processor, or the like connected to the endoscope 1.

If the zoom lens frame 51 moves in the optical axis direction, the second actuator 4 is deformed and generates electric power. Thus, the electric power is, through the connection wire 80, transmitted to the position detection processing circuit 85. The position detection processing circuit 85, from an amount of the generated power of the transmitted power, detects a position of the zoom lens frame 51 in the optical axis direction, and transmits the detection result to the feedback circuit 100 which is a control means.

Similarly to the above-described connection wire 70, the connection wire 80 is not necessary to be the lead wire, but the connection wire can be, for example, formed of an electric pattern structure formed on the inner circumference surface 10n of the image capturing unit 10 by coating or etching.

Between the tip surface of the second supporting member 53 and the base end surface of the zoom lens frame 51, and in the vicinity of the inner circumference of the second actuator 4, two elastic members 7, for example, coil springs, are provided. Also, the elastic members 7 are not limited to the coil springs, but can be springs or rubber.

The elastic members 7, with the elastic members 6, return the position of the moved zoom lens frame 51 in the optical axis direction to the position before the movement. Specifically, elastic members 7 maintain the position of the zoom lens frame 51 in the optical axis direction of the time the electric power is not supplied to the first actuator 3.

Now, operation of thus structured endoscope 1 according to the embodiment will be described.

To move forward and reversely the lens frame 51 in order to obtain an enlarged or wide-angle subject image, first, by the operation part (not shown) of the endoscope 1 is operated, from the actuator drive circuit 75 through the connection wire 70, electric power is supplied to the first actuator 3.

Then, for example, to obtain a subject image of five lens magnifications, a predetermined electric power corresponding to the five lens magnifications is supplied to the first actuator 3 from the actuator drive circuit 75.

In response to the power supply, the first EPAM 3a of the first actuator 3 contracts. Then, the electrodes 3b and 3c also contract. Thus, the zoom lens frame 51 moves forward in the optical axis direction so that the magnification is to be five lens magnifications.

In response to the above, the second EPAM 4a of the second actuator 4 deforms, that is, extends. Then, the electrodes 4b and 4c also extend. After the extension, the second actuator 4 generates electric power. The generated electricity is transmitted through the connection wire 80 to the position detection processing circuit 85.

The position detection processing circuit 85, in response to the supply of the generated electricity, detects the position of the zoom lens frame 51. Then, the position detection result of the zoom lens frame 51 is transmitted to the feedback circuit 100 of the endoscope 1.

The feedback circuit 100, in response to the position detection result of the zoom lens frame 51, detects whether the position of the zoom lens frame 51 in the optical axis direction is at the position corresponding to the five lens magnifications. If the position of the zoom lens frame 51 has not reached to the position corresponding to the five lens magnifications, the feedback circuit 100 transmits a signal to increase the amount of power supply to the actuator drive circuit 75. Thus, the lens frame 51 moves to the predetermined position in the forward optical axis direction.

On the other hand, if the position of the zoom lens frame 51 has exceeded the position in the forward optical axis direction, the feedback circuit 100 transmits a signal to reduce the amount of power supply to the actuator drive circuit 75. Thus, the lens frame 51 moves back to the predetermined position in the backward optical axis direction.

Finally, in order to return the zoom lens frame 51 to the position before the electric power was supplied to the first actuator 3, by an operation of the operation part (not shown), the power supply to the first actuator 3 from the actuator drive circuit 75 is interrupted. Thus, the zoom lens 51 is, by the elastic members 6 and 7, smoothly returned to the position before the power supply, and the position is maintained.

As described above, in the endoscope according to the embodiment, the forward/reverse movement of the zoom lens frame 51 which supports the zoom lens group 35 provided in the image capturing unit 10 in the optical axis direction is carried out using the first actuator 3 which has the first EPAM 3a, the plus electrode 3b, and the minus electrode 3c.

Accordingly, the zoom lens frame 51 moves forward and reversely in the optical axis direction by only supplying the electric power to the first actuator 3, and can be driven with good operationality by the low-cost, small, and lightweight mechanism. Thus, the drive means of the zoom lens frame 51 can be realized by the low-cost, small, and lightweight mechanism.

Further, in the drive of the zoom lens frame 51, drive by a motor, gear, or the like is not included. Accordingly, a stable operation of the zoom lens frame 51 can be obtained and the quality of the image capturing unit 10 can be increased.

Further, using that the second actuator 4 which includes the second EPAM 4a, the plus electrode 4b, and the minus electrode 4c deforms in response to the forward/reverse movement of the zoom lens frame 51 and generates the electric power, the forward/reverse movement position of the zoom lens frame 51 in the optical axis direction can be readily detected.

Accordingly, by feeding back the position detection result to the electric power to be supplied to the first actuator 3, the zoom lens frame 51, that is, the zoom lens group 35 can be moved forward and reversely with high accuracy in position.

Hereinafter, modifications will be described. In the embodiment, the first actuator 3 includes the EPAM 3a and the electrodes 3b and 3c which extend or contract in accordance with an interruption of power supply, and the second actuator 4 includes the EPAM 4a and the electrodes 4b and 4c which generate electricity by deformation. However, the invention is not limited to the above, the first actuator 3 and the second actuator 4 can be formed of a same member. Thus, the production costs can be reduced.

Further, in the embodiment, between the first supporting member 52 and the lens sliding convex part 51t of the zoom lens frame 51, the first actuator 3 is provided and between the second supporting member 53 and the lens sliding convex part 51t of the zoom lens frame 51, the second actuator 4 is provided.

However, the invention is not limited to the above, between the first supporting member 52 and the lens sliding convex part 51t of the zoom lens frame 51, the second actuator 4 can be provided and between the second supporting member 53 and the lens sliding convex part 51t of the zoom lens frame 51, the first actuator 3 can be provided.

### Second Embodiment

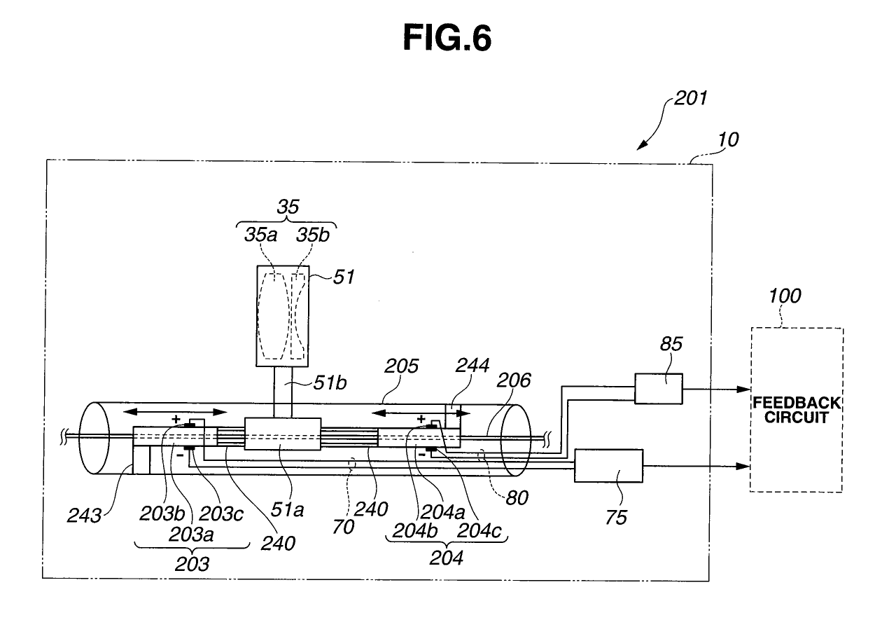

FIG. 6 is a view schematically illustrating a structure of a movement mechanism of a zoom lens frame of an image capturing unit provided at a tip of an insertion part of an endoscope illustrating the second embodiment of the present invention.

The structure of an endoscope 201 according to the embodiment, as compared with the endoscope 1 according to the first embodiment illustrated in FIGS. 1 to 5, differs in that a first actuator and a second actuator are provided in a movement drive mechanism of a zoom lens frame which moves by a seat. Accordingly, only the difference will be described. The same numbers are applied to similar structures to those in the first embodiment and those descriptions will be omitted.

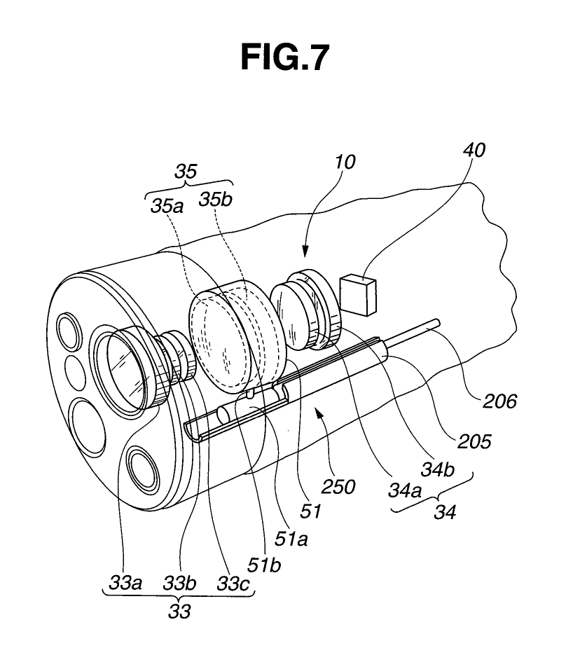

In advance of the description of the embodiment, a conventional structure that the zoom lens frame 51 is driven by a piezo element will be described. Specifically, in FIG. 7, a perspective view illustrating a conventional forward/reverse drive mechanism of a zoom lens frame is illustrated. As illustrated in the drawing, in the image capturing unit 10 and at lower part of the tip side lens group 33, the base end side lens group 34, and the zoom lens group 35, a drive unit 250 which is driven by a piezo element is provided.

In the drive unit 250, the main part is structured by a cylindrical member 205 which has an opening at an upper part and is provided along an optical axis direction, a rail 206 which passes through the cylindrical member 205, a seat 51a through which the rail 206 passes and is driven by the piezo element, and a leg part 51b in which one end is connected to the seat 51a and the other end is connected to the zoom lens frame 51 which holds the zoom lens group 35.

In thus structured drive unit 250, by the seat 51a, in the cylindrical member 205, by the piezo element, with only the leg part 51b is being exposed from the opening of the cylindrical member 205, moves forward and reversely in the optical axis direction, the zoom lens frame 51 connected to the seat 51a through the leg part 51b is moved forward and reversely in the optical axis direction by being guided by the rail 206.

The forward/reverse movement of the seat 51a is, in the embodiment, carried out using an actuator of an EPAM. Specifically, as illustrated in FIG. 6, to the tip surface of the seat 51a, a first actuator 203 is connected through a cylindrical connection member 240.

The first actuator 203 has a first EPAM 203a and a first electrode part which sandwiches at least a part of the first EPAM 203a, and has two electrodes, that is, a plus electrode 203b and a minus electrode 203c which are formed of a polymeric raw material, for example, a conductive rubber, and have different polar characters. The first actuator 203 is fixed to the cylindrical member 205 through a fixing member 243.

To the base end surface of the seat 51a, a second actuator 204 is connected through the cylindrical connection member 240. The second actuator 204 is fixed to the cylindrical member 205 through a fixing member 244.

The second actuator 204 has a first EPAM 204a and a second electrode part which sandwiches at least a part of the first EPAM 204a, and has two electrodes, that is, a plus electrode 204b and a minus electrode 204c which are formed of a polymeric raw material, for example, a conductive rubber, and have different polar characters.

The structures of the first actuator 203 and the second actuator 204 have substantially similar structures of the first actuator 3 and the second actuator 4 described in the above first embodiment, however, the actuators are formed of cylindrical members smaller in the diameters than those of the first actuator 3 and the second actuator 4.

The rail 206 is provided so as to pass through the seat 51a, the first actuator 203 and the second actuator 204. The other structures are similar to those in the endoscope 1 of the above-described first embodiment.

Now, operation of the thus structured endoscope 201 will be described.

To move forward and reversely the lens frame 51 in order to obtain an enlarged or wide-angle subject image, first, by the operation part (not shown) of the endoscope 201 is operated, from the actuator drive circuit 75 through the connection wire 70, electric power is supplied to the first actuator 203. Then, for example, to obtain a subject image of five lens magnifications, a predetermined electric power corresponding to the five lens magnifications is supplied to the first actuator 203 from the actuator drive circuit 75.

In response to the power supply, the first EPAM 203a which constitutes the first actuator 203 contracts. Then, the electrodes 203b and 203c also contract. Thus, the seat 51a connected to the first actuator 203 through the connection member 240 moves forward in the optical axis direction so that the magnification is to be five lens magnifications. Thus, the zoom lens frame 51 moves forward in the optical axis direction so that the magnification is to be five lens magnifications.

In response to the above, the second EPAM 204a of the second actuator 204 connected to the seat 51a through the connection member 240 deforms, that is, extends. Then, the electrodes 204b and 204c also extend. After the extension, the second actuator 4 generates electric power. The generated electricity is transmitted through the connection wire 80 to the position detection processing circuit 85. The other operations are similar to those in the endoscope 1 of the above-described first embodiment.

As described above, in the endoscope 201 according to the embodiment, the forward/reverse movement of the zoom lens frame 51 which has the structure to be driven forward and reversely by the piezo element is carried out using the two actuators 203 and 204 which have the EPAMs instead of the above-described piezo elements respectively. In such a drive mechanism, the zoom lens frame 51 moves forward and reversely in the optical axis direction by only supplying the electric power to the first actuator 203. Accordingly, the zoom lens frame 51 can be driven with good operationality by the low-cost, small, and lightweight mechanism. Thus, the drive means of the zoom lens frame 51 can be realized by the low-cost, small, and lightweight mechanism.

Further, using that the second actuator 204 deforms in response to the forward/reverse movement of the zoom lens frame 51, and generates the electric power, the forward/reverse movement position of the zoom lens frame 51 in the optical axis direction can be readily detected.

The other advantages, modifications are similar to those in the above-described first embodiment.

### Third Embodiment

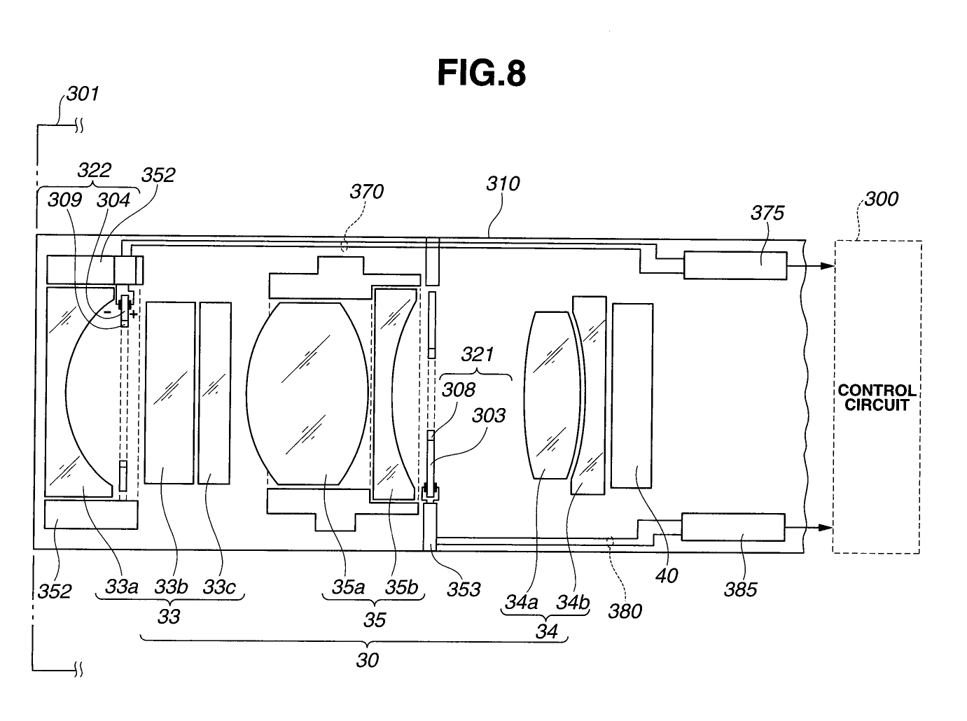

FIG. 8 is a view schematically illustrating a structure of an image capturing unit provided at a tip of an insertion part of an endoscope illustrating the third embodiment of the present invention.

A structure of an endoscope 301 according to the embodiment, as compared with the endoscope 1 of the first embodiment illustrated in FIGS. 1 to 5, differs in that a first actuator and a second actuator are provided in an aperture for depth adjustment and brightness adjustment, and an aperture for light shielding. Accordingly, only the difference will be described. The same numbers are applied to similar structures to those in the first embodiment and those descriptions will be omitted.

As illustrated in FIG. 8, in an image capturing unit 310 of the endoscope 301, the image capturing optical system 30 which includes the plurality of optical lenses is provided. At a base end side of the image capturing optical system 30, the image capturing element 40 such as a CCD on which a subject image received by the image capturing optical system 30 is formed is provided.

The image capturing optical system 30 includes the tip side lens group 33 which is provided to the tip side of the image capturing unit 10, the base end lens group 34 which is provided at the base end side of the image capturing unit 10 and in the vicinity of the tip of the image capturing element 40, and the zoom lens group 35 which is provided between the tip side lens group 33 and the base end lens group 34.

The tip side lens group 33 is the fixed lens system which includes, for example, the plurality of optical lenses 33a, 33b, and 33c, and the optical lenses 33a, 33b, and 33c are held by the lens frame (not shown) fixed to the tip side of the image capturing unit 10.

The base end lens group 34 is the fixed lens system which includes, for example, the plurality of optical lenses 34a and 34b, and the optical lenses 34a and 34b are held by the lens frame (not shown) fixed to the base end side of the image capturing unit 10.

The zoom lens group 35 includes, for example, the plurality of zoom lenses 35a and 35b. The zoom lens group 35 is held by the zoom lens frame 51, and by the above-described first actuator 3 (see FIG. 1), for example, moves forward and reversely between the tip side lens group 33 and the base end side lens group 34 in the optical axis direction.

At the back of the zoom lens frame 51 and in the vicinity of the base end side of the optical lens 35b, a lattice-shaped first supporting member 353 which is fixed to an inner circumference of the image capturing unit 310 is provided. Further, in the inner circumference of the first supporting member 353, a first aperture 321 is provided for depth adjustment and brightness adjustment of a subject image which contracts when electric power is supplied.

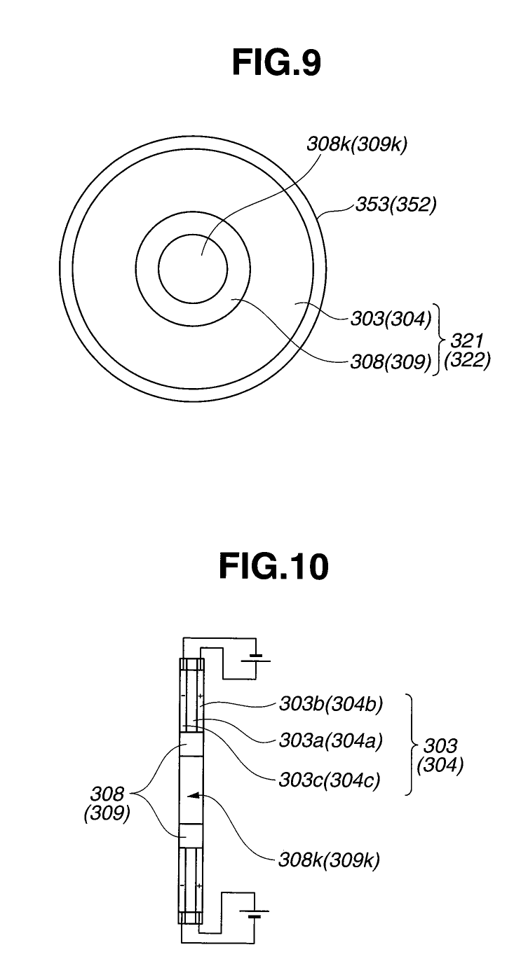

Specifically, FIG. 9 is an enlarged front view of the first aperture of FIG. 8, and FIG. 10 is a cross sectional view illustrating a structure of a first actuator 303 of the first aperture of FIG. 8. As illustrated in FIG. 9, in the first aperture 321, in the inner circumference of the substantially ring-shaped first actuator 303, a ring-shaped member 308 is fitted which has an opening 308k formed of a member harder than that of the first actuator, for example, a rubber.

Further, as illustrated in FIG. 10, the first actuator 303 includes a ring-shaped EPAM 303a which extends or contracts in accordance with an interruption of power supply, and a first electrode part which sandwiches the first EPAM 303a, and has two electrodes, that is, a plus electrode 303b and a minus electrode 303c which are formed of a polymeric raw material, for example, a conductive rubber, and have different polar characters.

Accordingly, the first actuator 303 is so called a polymer actuator. The first actuator 303 has a characteristic to extend if the actuator has contracted when the power supply is interrupted. A rate of contraction of the first EPAM 303a and rates of contraction of the plus electrode 303b and the minus electrode 303c are substantially the same.

To the plus electrode 303b and the minus electrode 303c, through a connection wire 380 which is a first signal wire of a lead wire, for example, electric power is supplied from a first actuator drive circuit 385 which is a power supply means provided at the base end side of the image capturing unit 310.

The connection wire 380 is provided in the vicinity of the inner circumference surface of the image capturing unit 310. One end of the connection wire 380 is electrically connected to the electrodes 303b and 303c, and the other end is connected to the first actuator drive circuit 385.

The first actuator drive circuit 385, in response to a signal from a control circuit 300 of the endoscope 301, supplies electric power to the first actuator 303. The first actuator drive circuit 385 can be provided in an operation part (not shown) of the endoscope 301, or a video processor, or the like connected to the endoscope 301.

Thus, because the first EPAM 303a contracts in the inner circumference direction, the first ring-shaped member 308 which is fitted into the first actuator 303 contracts in the inner circumference direction. That is, a diameter of the opening 308k of the first ring-shaped member 308 becomes a small diameter.

The connection wire 380 which transmits the electric power to the plus electrode 303b and the minus electrode 303c from the first actuator drive circuit 385 is not necessary to be the lead wire, but the connection wire can be, on the inner circumference of the image capturing unit 310, formed in an electric pattern structure by coating or etching. Thus, the inner structure of the image capturing unit 310 can be simplified.

In the vicinity of the outer circumference of the optical lens 33a of tip side lens group 33, a cylindrical second supporting member 352 is provided. Further, on the inner circumference of the second supporting member 352, a second aperture 322 for shielding which operates in conjunction with the first aperture 321 which contracts when electric power is supplied is provided.

Specifically, as illustrated in FIG. 9, in the second aperture 322, in an inner circumference of a substantially ring-shaped second actuator 304, a ring-shaped member 309 is fitted which has an opening 309k formed of a member harder than that of the second actuator 304, for example, a rubber.

Further, as illustrated in FIG. 10, the second actuator 304 includes a ring-shaped second EPAM 304a which extends or contracts in accordance with an interruption of power supply, and a second electrode part which sandwiches the second EPAM 304a, and has two electrodes, that is, a plus electrode 304b and a minus electrode 304c which are formed of a polymeric raw material, for example, a conductive rubber, and have different polar characters.

Accordingly, the second actuator 304 is so called a polymer actuator. The second actuator 304 also has a characteristic to extend if the actuator has contracted when the power supply is interrupted. A rate of contraction of the second EPAM 304a and rates of contraction of the plus electrode 304b and the minus electrode 304c are substantially the same.

To the plus electrode 304b and the minus electrode 304c, through a connection wire 370 which is a second signal wire of a lead wire, for example, electric power is supplied from a second actuator drive circuit 375 which is a power supply means provided at the base end side of the image capturing unit 310.

The connection wire 370 is provided in the vicinity of the inner circumference surface of the image capturing unit 310, one end of the connection wire 370 is electrically connected to the electrodes 304b and 304c, and the other end is connected to the second actuator drive circuit 375.

The second actuator drive circuit 375, in response to the reception of the signal from the control circuit 300 of the endoscope 301, supplies electric power to the second actuator 304. The second actuator drive circuit 375 can be provided in an operation part (not shown) of the endoscope 301, or a video processor, or the like connected to the endoscope 301.

Thus, the second EPAM 304a contracts in the inner circumference direction and the second ring-shaped member 309 fitted into the second actuator 304 contracts in the inner circumference direction. That is, a diameter of the opening 309k of the second ring-shaped member 309 becomes small.

The connection wire 370 which transmits the electric power to the plus electrode 304b and the minus electrode 304c from the second actuator drive circuit 375 is not necessary to be the lead wire, but the connection wire can be formed of an electric pattern structure formed on the inner circumference surface of the image capturing unit 310 by coating or etching. Thus, the inner structure of the image capturing unit 310 can be simplified.

Now, operation of thus structured endoscope 301 according to the embodiment will be described.

To adjust a depth or brightness of an image of a subject, first, by the operation part (not shown) of the endoscope 1 is operated, from the first actuator drive circuit 385 through the connection wire 380, to the first actuator 303 of the first aperture 321, a desired f-number, that is, a predetermined amount of electric power to obtain the desired brightness and the depth of field is provided.

In response to the power supply, the first EPAM 303a of the first actuator 303 contracts, for example, in the inner circumference direction. Then, the electrodes 303b and 303c also contract in the inner circumference direction.

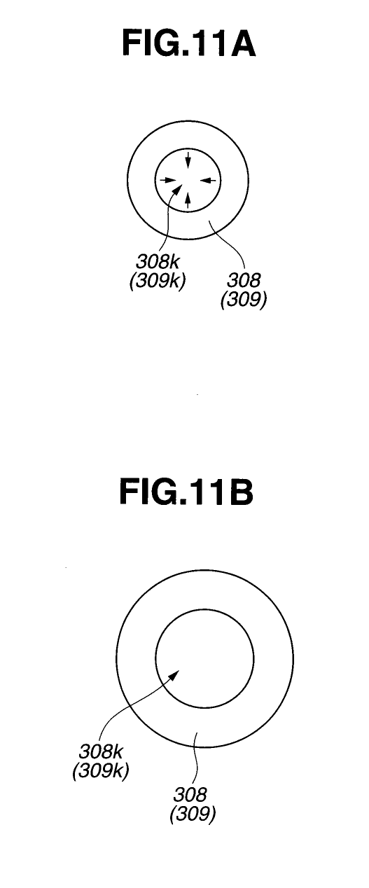

In response to the above, as illustrated in FIG. 11, the first ring-shaped member 308 fitted into the inner circumference of the first actuator 303 contracts in the inner circumference direction from a state of FIG. 11B to a state of FIG. 11A. That is, the diameter of the opening 308k of the first ring-shaped member becomes small.

Thus, the first aperture 321 is reduced and as well as ordinary apertures, the depth or brightness of the subject image is adjusted. Whether the diameter of the opening 308k of the first ring-shaped aperture is adjusted to the aperture value of the desired f-number is measured by a photometric sensor (not shown), for example, provided in an insertion part of the endoscope 301.

If the diameter of the opening 308k does not reach to the aperture value of the desired f-number, from the first actuator drive circuit 385 through the connection wire 380, electric power is further supplied to the first actuator 303, and in response to the contraction of the first EPAM 303a, the diameter of the opening 308K is further reduced.

If the diameter of the opening 308k exceeds the aperture value of the desired f-number, by reducing the amount of power supply from the first actuator drive circuit 385 through the connection wire 380 to the first actuator 303, the diameter of the opening 308K is increased to the desired position with the extension of the first EPAM 303a.

In conjunction with the operation of reducing the first aperture 321, from the second actuator drive circuit 375 through the connection wire 370 to the second actuator 304 of the second aperture 322, electric power is supplied.

In response to the power supply, the second EPAM 304a of the second actuator 304 contracts, for example, in the inner circumference direction. Then, the electrodes 304b and 304c also contract in the inner circumference direction.

In response to the above, as illustrated in FIG. 11, the second ring-shaped member 309 fitted into the inner circumference of the second actuator 304 also contracts in the inner circumference direction. That is, the diameter of the opening 309k of the second ring-shaped member becomes small. The diameter of the opening 309k is defined depending on the opening diameter of the opening 308k. Thus, the size of the second aperture 322 is reduced and unnecessary light to the first aperture 321 is shielded.

Finally, if the power supply to the first actuator 303 from the first actuator drive circuit 385 is stopped, the first EPAM 303a and the electrodes 303b and 303c extend, and as illustrated in FIG. 11B, the opening 308k of the first ring-shaped member 308 returns the position before the electric power supply.

At the same time, if the power supply to the second actuator 304 from the second actuator drive circuit 375 is stopped, the second EPAM 304a and the electrodes 304b and 304c extend, and as illustrated in FIG. 11B, the opening 309k of the second ring-shaped member 309 returns the position before the electric power supply.

As described above, in the endoscope according to the embodiment, the first aperture 321 for adjusting the depth and brightness of the subject image is structured by the first actuator 303 which includes the first EPAM 303a, the electrodes 303b and 303c. Further, the second aperture 322 for light shielding in conjunction with the aperture 321 is structured by the second actuator 304 which includes the second EPAM 304a, the electrodes 304b and 304c.

Accordingly, in the first aperture 321 and the second aperture 322, the apertures can be driven by only supplying electric power to the first actuator 303 and the second actuator 304 and can be driven with good operationality by the low-cost, small, and lightweight mechanism. Thus, the drive means of the first aperture 321 and the second aperture 322 can be realized by the low-cost, small, and lightweight mechanism.

Further, in the drive of the first aperture 321 and the second aperture 322, drive by a motor, gear, or the like is not included. Accordingly, a stable operation of the first aperture 321 and the second aperture 322 can be obtained and the quality of the image capturing unit 10 can be increased.

Hereinafter, modifications will be described. In the embodiment, the first actuator 303 includes the EPAM 303a, and the electrodes 303b and 303c, and the second actuator 304 includes the EPAM 304a, and the electrodes 304b and 304c. However, the invention is not limited to the above, the first actuator 3 and the second actuator 4 can be formed of a same member. Thus, the production costs can be reduced.

Generally, to the tip of the endoscope insertion part, an end cap which has a protrusion part of a horseshoe shape cross section is attached and fixed. The end cap, by maintaining an observation distance between the insertion part tip and a subject part to be constant, prevents the tip of the endoscope insertion part from abutting on a wall of the subject part, for example, to be focus failure and a field to the subject part cannot be ensured, and obserbility in a body cavity is increased.

The end cap is, generally, a part separated from the endoscope insertion part, and sometimes fixed to the insertion part tip with a tape, for example. However, in such a case, the protrusion amount of the protrusion part of the end cap differs depending on the condition of the fixation to the insertion part tip, and sometimes focus failure occurred in the subject part.

Moreover, the end cap is also used to facilitate the insertion into the insertion part. However, because of difficulty in attachment and detachment, fall, or difficulty in positioning, the range of fields can be interrupted.

In view of the above, a technique to integrate the end cap with the endoscope insertion part has been known. However, if the end cap is integrated, the protrusion amount of the protrusion part of the end cap cannot be changed. This is because that the shape of the subject part is not always plane, and if the amount of the protrusion part of the end cap is constant, it is difficult to always obtain a focused good image.

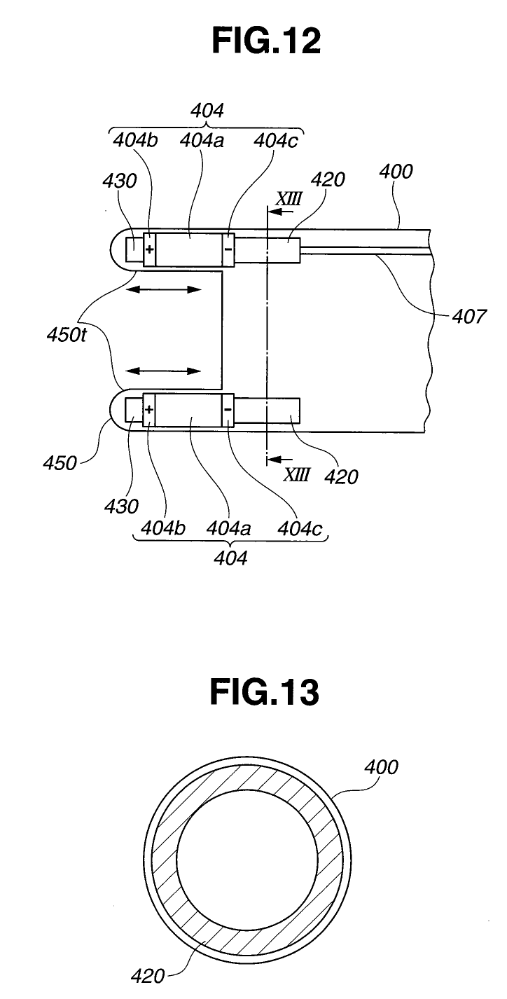

In view of the above, in the protrusion part of the end cap, an actuator formed of an EPAM can be provided. Specifically, in FIG. 12, a view illustrating a structure of the end cap attached to the endoscope insertion part tip is illustrated, and in FIG. 13, a cross sectional view taken along the line XIII-XIII of FIG. 12 is illustrated. As illustrated in FIG. 12, an end cap 450 has a protrusion part 450t, is formed of an elastic insulating member, and integrally formed by being coated to an tip part 400 of the endoscope insertion part.

To the tip part 400, a ring-shaped power supply ring member 420 illustrated in FIG. 13 connected to a power supply means (not shown) with a cable 407 is provided. Moreover, to the tip of the protrusion part 450t of the end cap 450, a ring-shaped power supply ring member 430 connected to a power supply means (not shown) with the cable 407 is provided.

Between the power supply ring member 420 of the end cap 450 and the power supply ring member 430, a ring-shaped actuator 404 which extends or contracts in accordance with an interruption of power supply is provided.

The actuator 404 includes a ring-shaped EPAM 404a and a first electrode part which sandwiches before and after the EPAM 404a in the optical axis direction, and has two electrodes, that is, a plus electrode 404b and a minus electrode 404c which are formed of a polymeric raw material, for example, a conductive rubber, and have different polar characters.

The structure of the actuator 404 is substantially similar to those of the first actuator 303 and the second actuator 304 in the above-described third embodiment.

The plus electrode 404b of the actuator 404 abuts on the power supply ring member 430 and the minus electrode 404c abuts on the power supply ring member 420. To the plus electrode 404b, electric power is supplied from the power supply ring member 430 and to the minus electrode 404c, electric power is supplied from the power supply ring member 420.

Now, operation of thus structured end cap 450 will be described.

To contract the protrusion part 450t of the end cap 450 in the optical axis direction base end side in order to focus, first, by the operation part (not shown) of the endoscope is operated, from the power supply means through the cable 407, power supply ring members 420 and 430, and the electrodes 404b and 404c to the EPAM 404a of the actuator 404, electric power is supplied.

In response to the power supply, the EPAM 404a of the actuator 404 contracts to the base end side in the drawing. Then, the electrodes 404b and 404c also contract to the base end side. Then, the protrusion part 450t of the end cap 450 contracts to the base end side.

Finally, if the power supply to the actuator 404 is stopped, the EPAM 404a of the actuator 404 extends to the tip side in the drawing. Then, the electrodes 404b and 404c also extend to the tip side. Then, the protrusion part 450t of the end cap 450 extends to the tip side, and return to the position before the power supply.

Thus, the protrusion amount of the protrusion part 450t of the end cap 450 can be varied by controlling the power supply to the EPAM 450a, and without providing a complicated mechanism, the protrusion amount of the protrusion part 450t cab be readily adjusted.

Further, since the end cap 450 and the end part 400 of the insertion part are integrally formed, the end cap 450 is prevented from falling off the end part 400. Moreover, since the positions where the end cap 450 is to be attached do not differ, the end cap 450 is prevented from interrupting the range of fields.

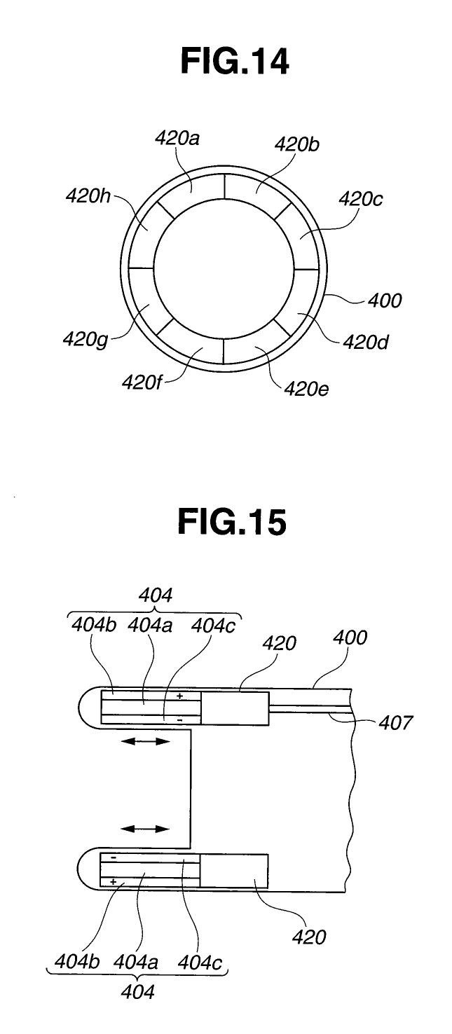

Hereinafter, a modification will be described. The power supply ring 420, as illustrated in FIG. 14, can be formed of a plurality of regions 420a to 420h and depending on each area, the protrusion amount of the protrusion part 450t can be adjusted.

Thus, the protrusion amount of the protrusion part 450t can be partially changed, that is, the degree of hardness of the elastic insulating member of the protrusion part 450t can be partially changed, and the appearance shape of the end cap can be readily adjusted to a shape according to a hardness distribution by only adjusting the power supply to the EPAM 404a.

Accordingly, even if the subject part has a complicated shape, since the protrusion part 450t of the end cap 450 can be fitted to the shape of the subject part, a focused good observation can be carried out.

Hereinafter, another modification will be described. It has been described that the plus electrode 404b and the minus electrode 404c of the actuator 404 sandwiches before and after the ring-shaped EPAM 404a in the optical axis direction. However, the present invention is not limited to the above, as illustrated in FIG. 15, the electrodes can be provided so as to sandwich the EPAM 404a in the thickness direction.

Thus, only one power supply ring member for supplying electric power to the electrodes 404b and 404c is necessary and the diameter of the tip part 400 of the insertion part can be reduced in diameter.

Although the embodiments of the present invention have been described above, the invention is not limited to the above embodiments, but various modifications can be made without departing from the spirit of the invention.

[Appendix]

As described in detail above, according to the embodiments of the present invention, the following structures can be obtained. That is,

(1) An endoscope comprising:

a plurality of lenses; and

a first aperture for depth adjustment and brightness adjustment of a subject image entering into the plurality of lenses,

wherein the first aperture comprises a first ring-shaped member and a first actuator provided on an outer circumference of the first ring-shaped member and changes an opening diameter of the first ring-shaped member by extending or contracting in accordance with an interruption of power supply.

(2) The endoscope according to appendix 1, wherein the first actuator comprises:

a first polymeric material which extends or contracts in accordance with the interruption of power supply; and

a first electrode part which includes two electrodes for sandwiching the first polymeric material and have different polar characters.

(3) The endoscope according to appendix 2, wherein the ring-shaped member is formed of a member harder than the first polymeric material.

(4) The endoscope according to any one of appendixes 1 to 3, the endoscope further comprises a second aperture for shielding the light entering into the plurality of lenses, wherein the second aperture comprises:

a second ring-shaped member; and

a second actuator provided on an outer circumference of the second ring-shaped member and changes an opening diameter of the second ring-shaped member by extending or contracting in accordance with an interruption of power supply.

(5) The endoscope according to appendix 4, wherein the second actuator comprises:

a second polymeric material which extends or contracts in accordance with the interruption of power supply; and

a second electrode part which includes two electrodes for sandwiching the second polymeric material and have different polar characters.

(6) The endoscope according to appendix 5, wherein the second ring-shaped member is formed of a member harder than the second polymeric material.

(7) The endoscope according to any one of appendixes 4 to 6, the endoscope further comprises power supply means for supplying electric power to the first actuator or the second actuator.

(8) The endoscope according to appendix 7, wherein the power supply means and the first electrode part are connected with each other with a first signal line.

(9) The endoscope according to appendix 8, wherein the first signal line is an electric pattern formed on the image capturing unit.

(10) The endoscope according to any one of appendixes 7 to 9, wherein the power supply means and the second electrode part are connected with each other with a second signal line.

(11) The endoscope according to appendix 10, wherein the second signal line is an electric pattern formed on the image capturing unit.

(12) The endoscope according to any one of appendixes 4 to 1, wherein the first actuator and the second actuator are formed of a same member.

(13) The endoscope according to any one of appendixes 4 to 12, wherein the second actuator contracts in conjunction with the first actuator.

(14) The endoscope according to any one of appendixes 4 to 13, wherein the opening diameter of the second ring-shaped member is defined depending on the opening diameter of the first ring-shaped member.

Problems to be Solved by the Invention

Endoscopes have been known which have an aperture for depth adjustment and brightness adjustment of a subject image provided in an image capturing optical system arranged at a tip part of an endoscope insertion part. The aperture for depth adjustment and brightness adjustment is generally provided as a fixed aperture to prevent the insertion part tip from being increased in its size and to simplify the structure.

However, in such a case, because the f-number of the aperture is fixed, the brightness of the subject is limited. Moreover, because the depth of the field is limited, the observable range is limited.

In view of the above, endoscopes provided with an aperture in which a depth of the field can be varied have been proposed. However, if the aperture variable mechanism is arranged at the insertion part tip, the size of the insertion part increases and the structure becomes complicated.

The appendixes have been made in view of the above, and it is an object to provide an endoscope capable of varying a f-number of an aperture with a simple structure and includes an aperture mechanism capable of controlling a depth of the field and brightness.

Although the embodiments of the present invention have been described above, the invention is not limited to the above embodiments, but various modifications can be made without departing from the spirit of the invention.

What is claimed is:

1. An endoscope comprising:
an image capturing unit which is provided with at least a plurality of lenses, a zoom lens frame for supporting lenses for zooming out of the plurality of lenses, and drive means for moving forward and reversely the zoom lens frame in an optical axis direction,
wherein the drive means includes a first actuator which extends or contracts in accordance with an interruption of power supply.

2. The endoscope according to claim 1, wherein the first actuator comprises:
a first polymeric material which extends or contracts in accordance with the interruption of power supply; and
a first electrode part which includes two electrodes for sandwiching the first polymeric material and have different polar characters.

3. The endoscope according to claim 1, further comprising position detection means for detecting a movement position of the zoom lens frame.

4. The endoscope according to claim 2, further comprising position detection means for detecting a movement position of the zoom lens frame.

5. The endoscope according to claim 3, wherein the position detection means includes a second actuator which generates electric power by deformation.

6. The endoscope according to claim 4, wherein the position detection means includes a second actuator which generates electric power by deformation.

7. The endoscope according to claim 5, wherein the second actuator comprises:
a second polymeric material which generates electric power by deformation; and
a second electrode part which includes two electrodes for sandwiching the second polymeric material and have different polar characters.

8. The endoscope according to claim 6, wherein the second actuator comprises:
a second polymeric material which generates electric power by deformation; and
a second electrode part which includes two electrodes for sandwiching the second polymeric material and have different polar characters.

9. The endoscope according to claim 7, wherein the position detection means detects the movement position of the zoom lens frame by detecting an amount of the generated power of the second polymeric material.

10. The endoscope according to claim 8, wherein the position detection means detects the movement position of the zoom lens frame by detecting an amount of the generated power of the second polymeric material.

11. The endoscope according to claim 9, further comprising:
control means for performing a control to define a position of the zoom lens frame in an optical axis direction based on the position detection result of the position detection means.

12. The endoscope according to claim 10, further comprising:
control means for performing a control to define a position of the zoom lens frame in an optical axis direction based on the position detection result of the position detection means.

13. The endoscope according to claim 11, wherein the control means and the first electrode part are connected by a first signal line.

14. The endoscope according to claim 12, wherein the control means and the first electrode part are connected by a first signal line.

15. The endoscope according to claim 13, wherein the first signal line is an electric pattern formed on the image capturing unit.

16. The endoscope according to claim 14, wherein the first signal line is an electric pattern formed on the image capturing unit.

17. The endoscope according to claim 11, wherein the control means and the second electrode part are connected by a second signal line.

18. The endoscope according to claim 12, wherein the control means and the second electrode part are connected by a second signal line.

19. The endoscope according to claim 13, wherein the control means and the second electrode part are connected by a second signal line.

20. The endoscope according to claim 14, wherein the control means and the second electrode part are connected by a second signal line.

21. The endoscope according to claim 15, wherein the control means and the second electrode part are connected by a second signal line.

22. The endoscope according to claim 16, wherein the control means and the second electrode part are connected by a second signal line.

23. The endoscope according to claim 17, wherein the second signal line is an electric pattern formed on the image capturing unit.

24. The endoscope according to claim 18, wherein the second signal line is an electric pattern formed on the image capturing unit.

25. The endoscope according to claim 19, wherein the second signal line is an electric pattern formed on the image capturing unit.

26. The endoscope according to claim 20, wherein the second signal line is an electric pattern formed on the image capturing unit.

27. The endoscope according to claim 21, wherein the second signal line is an electric pattern formed on the image capturing unit.

28. The endoscope according to claim 22, wherein the second signal line is an electric pattern formed on the image capturing unit.

29. The endoscope according to claim 5, wherein the first actuator and the second actuator are formed of a same member.
